(19) United States
(12) Patent Application Publication
(10) Pub. No.: US 2007/0123987 A1
(43) Pub. Date: May 31, 2007

(54) CURVILINEAR CERVICAL INTERBODY DEVICE

(76) Inventor: Avi J. Bernstein, Wilmette, IL (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 11/555,779

(22) Filed: Nov. 2, 2006

Related U.S. Application Data
(60) Provisional application No. 60/732,624, filed on Nov. 2, 2005.

(51) Int. Cl. A61F 2/44

(52) U.S. Cl. 623/17.11

(57) ABSTRACT

An interbody spacer assembly includes a pair of end pieces spaced apart by a connector extending between them. The end pieces extend generally parallel to the end plates of adjoining vertebral bodies. Fasteners connect the end pieces to the vertebral bodies. Bone graft material or solid bone can be placed in the interior space defined by the end pieces and connector, which bone graft material or solid bone eventually fuses together and to the adjoining end plates through the end pieces. The spacer assembly has ratchets to connect the two end pieces. The ratchets near the fasteners are more closely spaced than the ratchets further from the fasteners, allowing the spacer assembly to more closely approximate the lordosis of the spine.

### CROSS REFERENCE TO RELATED APPLICATIONS

This application claims priority of U.S. Provisional Patent Application No. 60/732,624, filed Nov. 2, 2005.

### FIELD OF THE INVENTION

This invention relates to cervical spine supports, and, in particular, to a device that acts as a spacer between cervical vertebral bodies so that bone graft material inserted within the device can fuse and replace pathological bone removed surgically.

### BACKGROUND

It is known in the prior art to use cage-like spacers made of titanium mesh in tube shapes between vertebrae to provide support to the cervical spine. Spacers are needed when either the vertebrae or disk are removed for pathological reasons due to injury or disease. The spacer maintains the granular bone tissue in place until the graft is complete. Some of the known prior art spacers, such as those described in application Ser. No. 10/293,843, which is incorporated by reference herein, may be difficult to install between existing vertebrae and difficult to satisfactorily fill with such bone tissue. Moreover, the cervical spacer as disclosed in application Ser. No. 10/293,843 may not correspond to the curvature of the cervical portion of the spine. In those locations along the spine where there is the most curvature, such as the neck and lower back, longitudinally straight spacers may fail or cause pain because they do not match or correspond to the natural curvature of the spine. This is particularly true when large sections of the vertebrae are replaced by a spacer because the curvature of a large section is greater than the curvature of a small section.

Consequently, I have developed a curvilinear cervical interbody device that is easier to install between cervical vertebral bodies, adjusts to the curvature of the cervical portion of the spine, can be readily adjusted to account for the size of the vertebrae or disks that are removed, and results in a stronger and more reliable graft.

### SUMMARY

A spacer assembly is provided for use in spinal surgeries. The spacer assembly comprises two end pieces for interfacing with the end plates of adjacent vertebrae. Each end piece is generally disk-like in form and includes an inner surface facing the interior of the spacer and an outer surface facing the adjacent vertebrae. Each end piece has attached thereto a flange that extends longitudinally when installed (i.e., in the general direction of the length of the spine) and exteriorly of the end piece. The end pieces are spaced and reinforced by one or more connectors. The spacer assembly engages the adjacent vertebral disks by securing each flange with the adjacent vertebrae to couple the assembly and vertebrae together. The spacer assembly defines an interior region that is filled with morselized bone graft, structural bone graft, biologic fusion materials, or solid bone to fuse together and with the adjacent vertebrae, thereby replacing pathological bone or disk material removed surgically. The spacer assembly can be adjusted by ratcheted connectors, with the ratchets preferably being distributed so that the assembly's radius corresponds approximately to the radius of the spine in the area of the removed vertebrae or disks even as the assembly increases or decreases in average size.

In one embodiment, the end pieces are contoured to conform to the cross-section shape of the spinal cord. The end pieces are further designed to promote bone growth into the adjacent areas by, for instance, including apertures or an opening between the interior region and the vertebrae.

The inventive spacer assembly can be used to replace either a surgically removed disk (diskectomy) or vertebra (corpectomy).

### BRIEF DESCRIPTION OF THE DRAWINGS

FIG. 1 is a side perspective view of the device in an extended position.

FIG. 2 is a side perspective view of the device in a contracted position.

FIG. 3 is a side perspective, cut-away view of the device implanted in the spine.

FIG. 4 is a perspective view of the disassembled device.

FIG. 5 is a perspective view of a second embodiment of the device which can be further extended than the first embodiment.

FIG. 6 is a side perspective, cut-away view of the second embodiment implanted in the spine.

FIGS. 7 and 7a are cross-sectional views showing the geometry of the intermeshing teeth of the ratchets.

### DETAILED DESCRIPTION

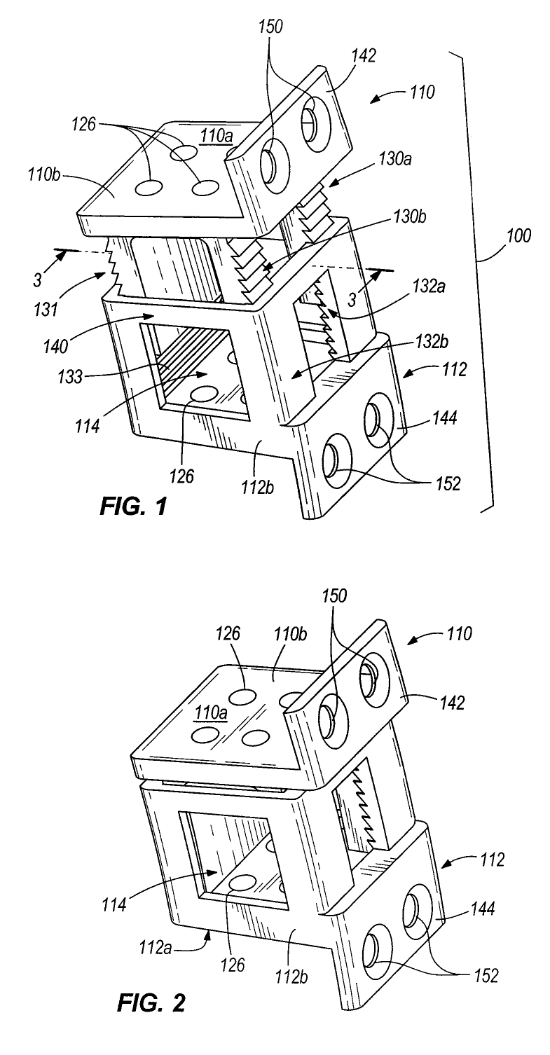

As seen in FIGS. 1 and 2, the spacer assembly 100 includes an upper end piece 110 and a lower end piece 112. End piece 110 comprises an exterior surface 110a in first end plate 110b, integrally formed flange 142 for attaching the assembly to a vertebral body, and stepped or ratcheting connectors 130a, 130b, 131. End piece 112 comprises an exterior surface 112a on second end plate 112b, integrally formed flange 144, and stepped or ratcheting connectors 132a, 132b, 133. End piece 112 is shown with an optional stabilizing piece 140 connecting ratcheting connectors 132a, 132b and 133, for instance by going around the perimeter of the end piece 112, providing additional structural integrity to the end piece. Thus, when the two end pieces are assembled, the tendency of the internal ratcheting connectors 130a, 130b, 131 to push out the external connectors 132a, 132b, 133 is minimized by the presence of the connecting piece 140.

In one embodiment, the ratcheting connectors 130, 132 are a pair of column-like parts, and the ratcheting connectors 131, 133 are wall-like, and extend the width of the spacer. While FIGS. 1 and 2 show the end piece 110 as having ratcheting connectors 130, 131 internal to the connectors 132, 133 of end piece 112, a variation in which the end piece 112 is internal to end piece 110 is also feasible. In such a variation, the connecting, stabilizing piece 140 would be on end piece 110.

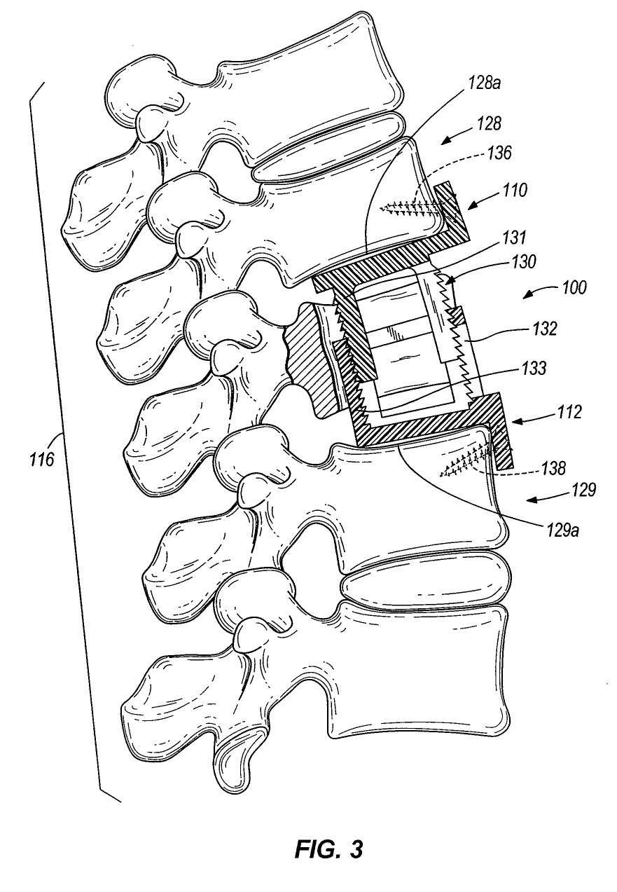

As seen in FIG. 3, which features a cross-section of the spacer assembly 100 taken through lines 3, the spacer assembly 100 in a collapsed state is located by a surgeon between the vertebral bodies of a spine 116, from which one or more diseased or damaged vertebrae or disks were removed during surgery. The spacer assembly 100 is then expanded to maintain the vertebrae in a spaced-apart configuration. The spacer assembly 100 is placed from the front of the patient, using an anterior approach, to fill up the entire disc space or replace the entire vertebral body or bodies, both longitudinally and laterally.

When the spacer assembly has been installed, the exterior surfaces 110a and 112a of the end pieces 110, 112 are substantially parallel to the adjoining surfaces 128a, 129a (often referred to as “end plates”) of the vertebral bodies 128, 129. The end pieces 110, 112 preferably have a substantially flat or planar outer surface to provide a stable interface with the end plates, and the end pieces may be shaped and dimensioned to closely match the cross-sectional shape and dimensions of the end plates.

The end pieces 110, 112 are adjustably connected to each other by their ratcheted connectors 130, 132 and 131, 133 so as to establish a desired length of the spacer assembly 100. The ratcheted connectors allow the spacer assembly to be extended or shortened to conform most closely to the space between the vertebral bodies 128, 129. By adjusting the ratcheting connectors for the desired spacing between the vertebral bodies, a surgeon can achieve optimal biomechanical strength in situ. The columnar ratcheted connectors 130a and 130b may be flexible enough to permit the surgeon to disengage them from their mating columnar ratcheted connectors 132a, 132b.

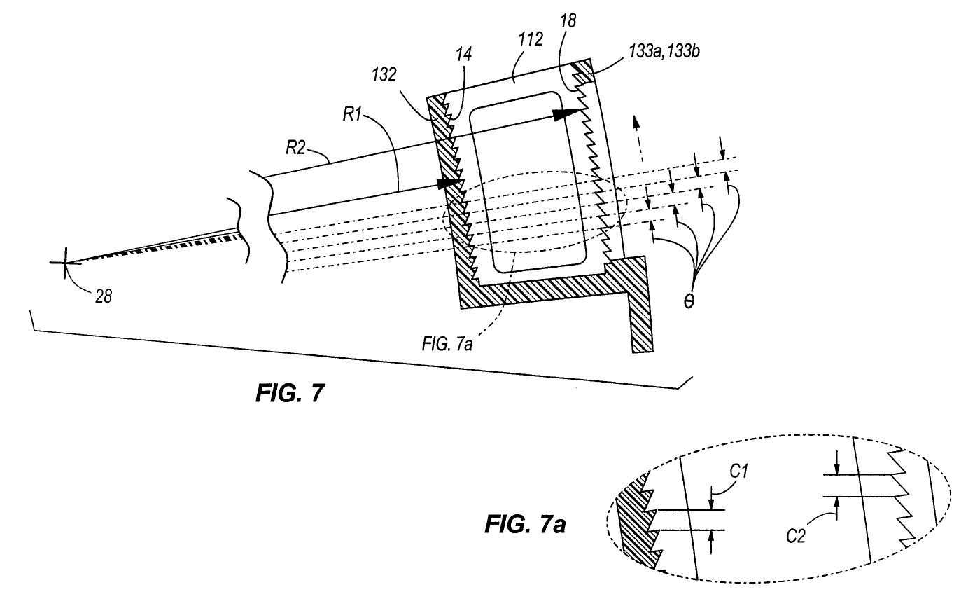

In addition, as described in more detail with respect to FIGS. 7 and 7a, the interdigitation of the teeth in the ratcheted connectors has been designed so that the ratcheted connectors 131, 133, which are nearer the spine, are relatively shorter than the corresponding ratcheted connectors 130, 132, which are further from the spine. As the spacer assembly is expanded, the lordosis or curvature of the assembly correspondingly increases. This results in a spacer assembly that more closely follows the lordosis of the spine in which it is placed.

The ratcheted connectors may be of equal length or they may be of different lengths. It is the curvature of the connector which determines the degree of lordosis. As the device is expanded, the degree of lordosis increases.

As shown in FIGS. 7 and 7a, the geometry of the intermeshing teeth of the ratchets may be established to account for the natural curvature as follows. With reference to FIGS. 10 and 10a, the end piece 112 includes forward teeth 14 formed on front wall 132 and rearward teeth 18 formed on rear columns 133a, 133b. When viewed as arcs of circles, the front wall 132 and rear columns 133a, 133b extend concentrically about center 28. The relative size between teeth 14 and teeth 18 corresponds to the sweep angle θ between adjacent teeth and the difference between R1 and R2. More specifically, tooth height C1 and C2 can be found using the following:

$$C 1 = 2 \cdot R 1 \cdot sin ( \frac{θ}{2} )$$

$$C 2 = 2 \cdot R 2 \cdot sin ( \frac{θ}{2} )$$

Thus, as the difference between R1 and R2 increases or decreases, the respective tooth heights will increase or decrease proportionally and according to the above formulae. The leading edge of each forward tooth 14 is thereby radially aligned with a corresponding leading edge of a rearward tooth 18. The number of teeth formed in the end piece 112 is dictated by the height of the end piece and the sweep angle θ between teeth. In other words, the assembly is designed so that the exterior surfaces 110a, 112a of the endpieces 110, 112 become less parallel as the assembly expands, and more parallel as it collapses so that the spacer assembly has a curvature that is similar to the curvature or lordosis of the spine. The posterior ratchets are more closely spaced, i.e., the ratchets are smaller, than the anterior ratchets, and thus C1>C2, so that as the device is lengthened, it does so in a curvilinear path or fashion.

The end pieces 110, 112 may be squarish or approximately disk-shaped to conform to the cross-sectional shape of the end plates of the adjacent vertebrae. The exterior surfaces 110a and 112a, respectively, of end pieces 110 and 112 interface with the end plates of adjacent vertebrae 128, 129. The portion of the end pieces surrounding the spinal cord are preferably contoured to avoid compressing or otherwise affecting the spinal cord.

The interior region 114 between end pieces 110, 112 is substantially open around its perimeter, and it can be easily filled with bone graft tissue to fuse to vertebral bodies 128, 129 of spine 116. The end pieces 110, 112 contain apertures 126 extending through their thickness to allow the bone graft tissue to grow through the end pieces and into the adjacent vertebrae, and thereby providing direct contact between the bone graft tissue and the adjoining vertebrae. Multiple apertures 126 are preferred to permit the bone graft tissue in region 114 to fuse with the adjacent vertebrae.

The end pieces 110, 112 have integrally formed flanges 142, 144 projecting approximately perpendicularly from the exterior surfaces 110a, 112a, respectively, and the flanges 142, 144 are located around the perimeter of a portion of the exterior surfaces 110a, 112a, respectively. The flanges act as stops to engage the assembly in proper position relative to the spine. They also prevent retropulsion or compression of the spinal cord, which can occur if the assembly were to slide too far into the spine toward the spinal cord 116 or otherwise shift out of place.

The flanges have holes 150, 152 for receiving screws 136, 138 of the type customarily used in spine surgeries. These screws 136, 138 are screwed into the adjacent vertebral bodies 128, 129 respectively, preferably with commonly available locking mechanisms, to secure the spacer assembly in place relative to the spine. Alternatively, screws could be located through apertures in the end pieces and directly into the vertebrae. Preferably, the screws are inserted through the flange at an angle toward or away from the adjoining end piece, rather than parallel thereto, to increase the stability of the device and reduce the possibility of inadvertent displacement.

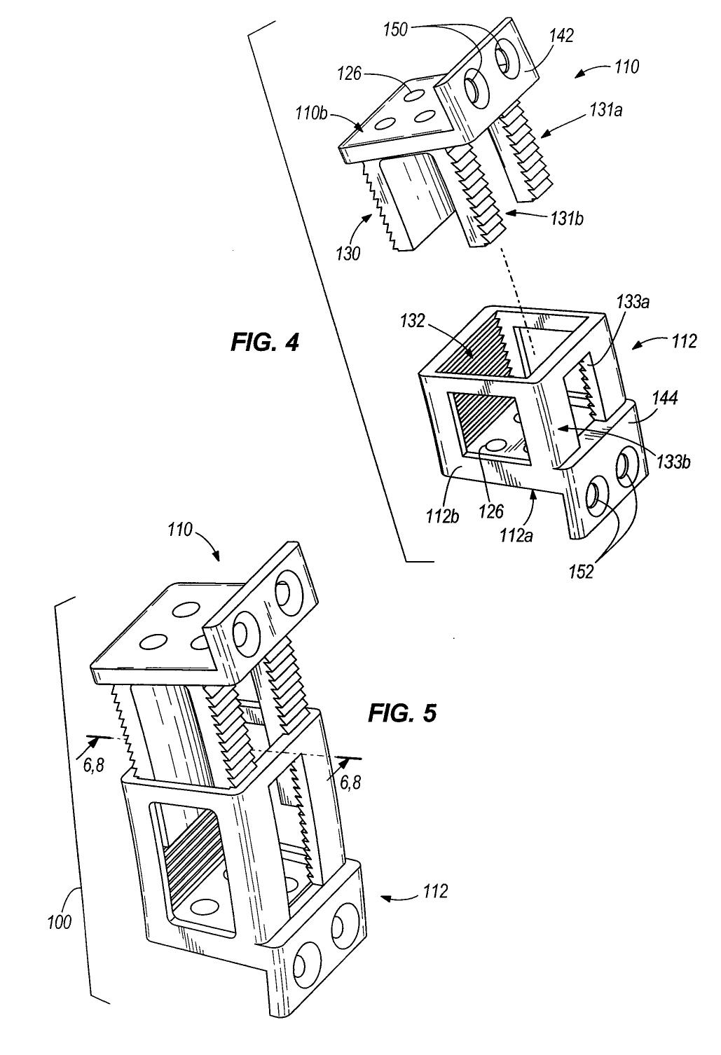

As seen in FIG. 4, the wall 130 of end piece 110 comprises a step-like structure, and the columns 131a, 131b comprise step-like structures. Likewise, the wall 132 of end piece 112 comprises a step-like structure, and the columns 133a, 133b also comprise step-like structures. As shown, end piece 110 fits within end piece 112, with the wall 130 and columns 131a, 131b interacting with wall 132, and columns 133a, 133b in stepwise fashion. The columns 131a, 131b, 133a, 133b may be somewhat flexible laterally (i.e., perpendicular to the spine) to permit disengagement and contraction or expansion by the surgeon if that is necessary. This flexibility can be accomplished by appropriately thinning the wall and columns or by providing slits in them to allow bending. Additionally, flexible material or a spring-like mechanism could be used.

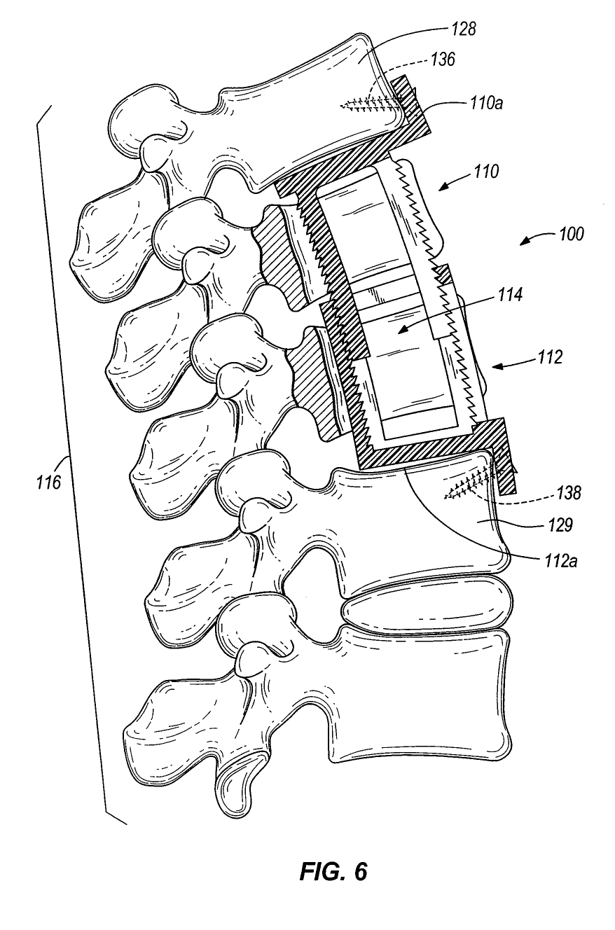

A second embodiment of the spacer assembly is shown in FIGS. 5-6. This device is similar to the first embodiment except that it is sized sufficiently to allow it to replace two vertebral bodies.

Not shown is a mesh, or retainer, that partially but does not entirely surround interior region 114 between the end pieces where the bone graft tissue is located and spans the distance between the end pieces and fills the interior region 114. This mesh is preferably located at the anterior side of assembly 100 and helps retain the bone graft tissue and prevent it from dislodging during implantation of the assembly. The mesh is held in place relative to the rest of assembly 110 by screws extending through the mesh, through holes 150, 152 of flanges 142, 144, and finally into the adjacent vertebrae. Thus, the mesh can be installed after the bone graft tissue is positioned.

The remaining region 114 is not surrounded by mesh because a patient's muscle tissue along the spine will partially enclose the area 114. Preferably, the mesh has an arcuate width that is slightly larger than the arcuate width of flanges 142, 144. The connector is located at the posterior side of the assembly, closest to the spinal cord, where it protects the spinal cord from the bone graft tissue. This embodiment can be supplemented with anteriorly-located connectors in the form of posts, if desired for additional strength.

Additionally, the exterior surfaces 110a and 112a of end pieces 110 and 112, respectively, may be roughened or formed with alternating ridges and valleys (not shown). The ridges are angled relative to the planes of surfaces 110a and 112a so that the peak of each ridge is on the anterior side (i.e. farthest from the spinal cord) of the ridge. Stated differently, the ridges are slanted so that the anterior side of each ridge forms an angle less than 90 degrees with the plane of the exterior surface of the end piece (e.g. 110a), while the posterior side of each ridge forms an angle greater than 90 degrees with the plane (e.g. 110a) of the exterior surface of the end piece. This arrangement permits the assembly 100 to easily slide laterally between the spaced vertebrae 128, 129, while also resisting lateral movement in the opposite direction away from the spaced vertebrae. This helps prevent inadvertent dislocation of the assembly away from the desired position between the vertebrae.

The end pieces and flanges are desirably composed of titanium or a bioabsorbable material, but they may also be composed of other rigid materials such as other metals and plastics. There is no need for adjuvant fixation, such as with a plate or another device to stabilize the position of the assembly. An acceptable plastic would be polyetheretherketone. Resorbable plates may also be used.

The present assembly has been described in connection with cervical vertebral bodies, but the same invention could be applied to the thoracic and lumbar spine by simply varying the shapes and dimensions of the components to correspond to the shapes and dimensions of the thoracic and lumbar vertebrae.

It should be recognized that, while the spacer assembly has been described in relation to a preferred embodiment, those skilled in the art may develop a wide variation of structural details without departing from the principles described here. Accordingly, the appended claims are to be construed to cover all equivalents falling within the scope and spirit of the disclosure.

The invention claimed is:

1. An interbody spacer assembly for replacing either a vertebra or disk, comprising:
first and second end pieces, said end pieces spaced apart from each other and defining an interior region between said end pieces for receiving bone graft material;
at least one fastener for securing the spacer assembly to a vertebral body; and
a curvilinear connector between the first and second end pieces to adjust the spacing between them.

2. The spacer assembly of claim 1 further comprising a retainer spanning said first and second end pieces for retaining bone graft material.

3. The spacer assembly of claim 1 and a flange extending from at least one of said end pieces, said flange receiving said at least one fastener.

4. The spacer assembly of claim 1 wherein the connector is a rod.

5. The spacer assembly of claim 1 wherein the connector is a wall.

6. The spacer assembly of claim 5 wherein the connector is positioned so that it can extend along the spinal cord.

7. The spacer assembly of claim 1 wherein the end pieces each have an end plate, and the end plates are substantially parallel when the spacing between them is minimized and progressively less parallel as the spacing increases.

8. The spacer assembly of claim 1 wherein said end pieces each have an outer surface that is substantially flat.

9. An interbody spacer assembly for replacing either a vertebra or a disk, comprising:
first and second end pieces, the end pieces spaced apart from each other and including mating connectors extending between the first and second end pieces;
at least one fastener for securing the spacer assembly to a vertebral body;
a flange extending from at least one of said end pieces, the flange receiving the at least one fastener for securing the spacer assembly to a vertebral body; and
ratchets on the connectors, the ratchets having a first spacing on the first end piece and a second spacing on the second end piece.

10. The spacer assembly of claim 9 wherein the first and second end pieces define an open interior region between them.

11. The spacer assembly of claim 9 wherein said end pieces each include a plurality of apertures.

12. The spacer assembly of claim 9 wherein the end pieces each have an outer surface that is substantially flat.

13. The spacer assembly of claim 9 wherein said end pieces each have a roughened exterior surface.

14. The spacer assembly of claim 12 wherein said roughened surfaces are comprised of alternating ridges and valleys.

15. The spacer assembly of claim 9 wherein said mating connectors of one of said first and second end pieces comprises a pair of arms defining a space between said arms, and wherein said space between said arms communicates with an open interior region between said end pieces.

16. An interbody spacer assembly for replacing either a vertebra or disk, comprising:
a pair of end pieces, each of said end pieces having a plurality of apertures extending through said end pieces;
a connector adjustably connecting and spacing said end pieces to correspond to a curvature of the replaced vertebra or disk, said end pieces and connector defining an interior space;
a flange extending from each of said end pieces and away from said interior space;
at least one fastener engaging at least one of said flanges for securing said spacer assembly to a vertebral body; and
a retainer extending between the first and second end pieces for retaining bone graft material within said interior space.

17. The interbody spacer assembly of claim 16 wherein said connector has intermeshing teeth.

18. The interbody spacer assembly of claim 17 wherein the intermeshing teeth are of different sizes.

19. The interbody spacer assembly of claim 16 wherein the intermeshing teeth nearer the flange are less closely spaced than the intermeshing teeth further from the flange.

20. The interbody spacer assembly of claim 16 wherein the end pieces each have an outer surface that is substantially flat.

21. A method of inserting an interbody spacer assembly for replacing vertebral bodies of a spine, said method comprising:
placing the interbody spacer assembly between vertebral bodies of a spine, wherein the interbody spacer assembly comprises first and second end pieces, said end pieces spaced apart from each other; and
moving the first and second end pieces relative to each other in a curvilinear path to substantially fill the space between the vertebral bodies both longitudinally and laterally.

22. The method of claim 21 further comprising placing the interbody spacer assembly from the front of a patient using an anterior approach.

23. The method of claim 21 further comprising fastening the interbody spacer assembly to adjacent vertebral bodies of a spine.

24. The method of claim 21 wherein the first and second end pieces move away from each other.
